(19) United States
(12) Patent Application Publication
(10) Pub. No.: US 2014/0212989 A1
(43) Pub. Date: Jul. 31, 2014

(54) PHENYTOIN BIOSENSOR AND METHOD FOR MEASURING CONCENTRATION OF PHENYTOIN

(71) Applicant: National Taiwan University, Taipei (TW)

(72) Inventors: Long-Sun Huang, Taipei (TW); Lung-Yi Lin, Taipei (TW); Yu-Chen Chang, Taipei (TW); Yotsapoom Pheanpanitporn, Taipei (TW)

(73) Assignee: National Taiwan University, Taipei (TW)

(21) Appl. No.: 13/956,257

(22) Filed: Jul. 31, 2013

(30) Foreign Application Priority Data
Jan. 28, 2013 (TW) 102103200

(51) Int. Cl. G01N 33/94; G01N 33/543

(52) U.S. Cl. CPC G01N 33/9473; G01N 33/5438
USPC 436/501; 422/69

(57) ABSTRACT

The present disclosure relates to a phenytoin biosensor. In some embodiments, the phenytoin biosensor may comprise a microcantilever, a self-assembly monolayer, and a phenytoin antibody layer. The self-assembly monolayer may immobilize on the microcantilever surface. The phenytoin antibody layer may immobilize on the self-assembly monolayer. The phenytoin antibody layer may be used to bind with phenytoin drug samples. The present disclosure further relates to methods for measuring the concentration of phenytoin drug samples.

### CROSS-REFERENCE TO RELATED APPLICATION(S)

This application claims priority to Taiwan Patent Application No. 102103200, filed on Jan. 28, 2013, the disclosure of which is hereby incorporated by reference in its entirety.

### FIELD OF THE DISCLOSURE

The present disclosure relates, in some embodiments, to a biosensor. More specifically, the present disclosure relates, in some embodiments, to a microcantilever biosensor.

### BACKGROUND OF THE DISCLOSURE

Phenytoin is one of the most widely used antiepileptic drugs. To be effective as a remedy, the concentration of phenytoin in the blood vessels must be kept within a suitable range. Ineffective treatment may occur if the treatment dosage is too low. Even worse, adverse effects may occur if the treatment dosage is too high. Therefore, monitoring the concentration of phenytoin in the blood vessels is very important.

The size of instruments used to monitor the concentration of phenytoin may be large. Thus, the monitoring instruments may not be portable and their prices may be very expensive. Consequently, patients cannot immediately determine whether or not the concentration of the drug in their blood vessels is within the optimal range for effective treatment.

### SUMMARY

Accordingly, there exists a need for an improved phenytoin biosensor that can address the aforementioned drawbacks.

The present disclosure relates, in some embodiments, to phenytoin biosensors and a methods for measuring the concentration of phenytoin in the blood vessels. Some embodiments of the present disclosure relate to phenytoin biosensors that may be small in size and may thus be portable for a point-of-care platform and personal diagnosis. As a result, patients may, anytime and anywhere, easily use the biosensor to assess their health and determine whether or not the concentration of phenytoin in their blood vessels is within the optimal range for effective treatment.

Some embodiments of the present disclosure relate to phenytoin biosensors that may be comprise a microcantilever, a self-assembly monolayer, and a phenytoin antibody layer. The self-assembly monolayer may be immobilized on the microcantilever surface. The phenytoin antibody layer may be immobilized on the self-assembly monolayer. The phenytoin antibody layer may be used to bind with phenytoin drug samples.

Some embodiments of the present disclosure relate to methods for measuring the concentration of phenytoin in blood vessel. A method may comprise: manufacturing a microcantilever with a piezoresistive layer; binding a plurality of self-assembly molecules to the microcantilever; activating the bonded self-assembly molecules; binding a plurality of phenytoin antibodies with the activated self-assembly molecules; binding a plurality of phenytoin drug samples with the phenytoin antibodies; measuring a change of resistance of the piezoresistive layer; and calculating the concentration of phenytoin according to the previously established relationship between the measured resistance change and the concentration of the phenytoin drug samples.

Some embodiments of the present disclosure relate to methods for measuring the concentration of phenytoin. The steps of the method may comprise: manufacturing a microcantilever with a field effect transistor; binding a plurality of self-assembly molecules to the microcantilever; activating the bonded self-assembly molecules; binding a plurality of phenytoin antibodies with the activated self-assembly molecules; binding a plurality of phenytoin drug samples with the phenytoin antibodies; measuring a change of current of the field effect transistor; and calculating the concentration of phenytoin according to the previously established relationship between the measured current change and the concentration of the phenytoin drug samples.

### BRIEF DESCRIPTION OF THE DRAWINGS

FIG. 1 illustrates a schematic view of a phenytoin biosensor according to some example embodiments of the disclosure;

FIG. 2 illustrates a flowchart of one embodiment of a method for measuring the concentration of phenytoin;

FIG. 3 illustrates a schematic view of self-assembly molecules bonded to a microcantilever surface according to some example embodiments of the disclosure;

FIG. 4 illustrates a schematic view of the activation of self-assembly molecules according to some example embodiments of the disclosure;

FIG. 5 illustrates a schematic view illustrating phenytoin antibodies bonded with self-assembly molecules according to some example embodiments of the disclosure;

FIG. 6 illustrates a schematic view of phenytoin antibodies bonded with phenytoin drug samples according to some example embodiments of the disclosure;

FIG. 7 illustrates changes in resistance and surface stress of a microcantilever after self-assembly molecules bind to a microcantilever according to some example embodiments of the disclosure;

FIG. 8 illustrates changes in resistance and surface stress of a microcantilever after phenytoin antibodies bind with the self-assembly molecules, according to some example embodiments of the disclosure;

FIG. 9 illustrates changes in resistance and surface stress of a microcantilever after phenytoin drug samples bind to the microcantilever according to some example embodiments of the disclosure;

FIG. 10 illustrates a phenytoin biosensor according to some example embodiments of the disclosure;

FIG. 11 illustrates a front view of the phenytoin biosensor shown in FIG. 10;

FIG. 12 illustrates a flowchart of an embodiment of a method for measuring the concentration of phenytoin according to some example embodiments of the disclosure;

FIG. 13 illustrates a schematic view of a phenytoin biosensor according to some example embodiments of the disclosure.

### DETAILED DESCRIPTION

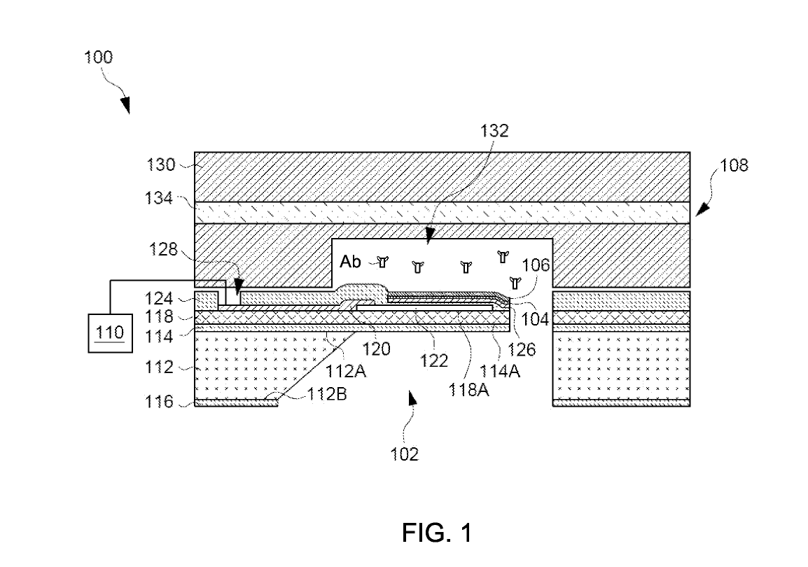

FIG. 1 is a schematic view illustrating a phenytoin biosensor 100 according to some example embodiments of the disclosure. As shown in FIG. 1, the phenytoin biosensor 100 may comprise a microcantilever 102, a self-assembly monolayer 104, and a phenytoin antibody layer 106, a microchannel 108, and a measuring equipment 110. The microcantilever 102 may include a substrate 112, which may be made of silicon. A passivation layer 114 may be deposited on a top surface 112A of the substrate 112, and a passivation layer 116 may be deposited on a bottom surface 112B of the substrate 112. The passivation layer 114 may also be used as a structural layer of the microcantilever. The passivation layers 114 and 116 may be made of Si₃N₄. A stress balance layer 118 may be deposited on a top surface 114A of the passivation layer 114. The stress balance layer 118 may be made of SiO₂. A conducting wire 120, a piezoresistive layer 122, and a passivation layer 124 may be on a top surface 118A of the stress balance layer 118. The conducting wire 120 may be made of Au and may contact the piezoresistive layer 122. The passivation layer 124 may be made of Si₃N₄ and may cover the conducting wire 120. There may be a hole 128 on one end of the passivation layer. One end of the conducting wire 120 may be exposed through the hole 128 and may connect with the measuring equipment 110. One end of the passivation layer 124 may be connected with the piezoresistive layer 122 and the stress balance layer 118. A sensing layer 126 may be deposited on a top surface of the passivation layer 124 and may be above the piezoresistive layer 122. The piezoresistive layer 122 may be made of polysilicon. The sensing layer 126 may be made of a gold film. In preferred embodiments, the thickness of the sensing layer 126 may be less than 100 nm. The microchannel 108 may include a top cover 130 and a channel 132. There may be a conductive glass layer 134 in the top cover 130.

The self-assembly monolayer 104 (SAM) may be composed of a plurality of self-assembly molecules which may be 8-Mercaptooctanoic acid. The self-assembly monolayer 104 may be formed by binding the self-assembly molecules to the sensing layer 126. The phenytoin antibody layer 106 may be composed of a plurality of phenytoin antibodies (Ab) and may be formed by binding the phenytoin antibodies with the self-assembly monolayer 104. The microcantilever 102 may be covered in the microchannel 108, and a plurality of phenytoin drug samples (Analyte) may be injected into the microchannel 108 to bind with the phenytoin antibodies. The measuring equipment 110 may connect with the conducting wire 120 and the piezoresistive layer 122. The measuring equipment may then be used to measure the change of the resistance of the piezoresistive layer 122 and to then determine the concentration of phenytoin based on the previously determined relationship between the change of the resistance and the concentration of the phenytoin drug samples.

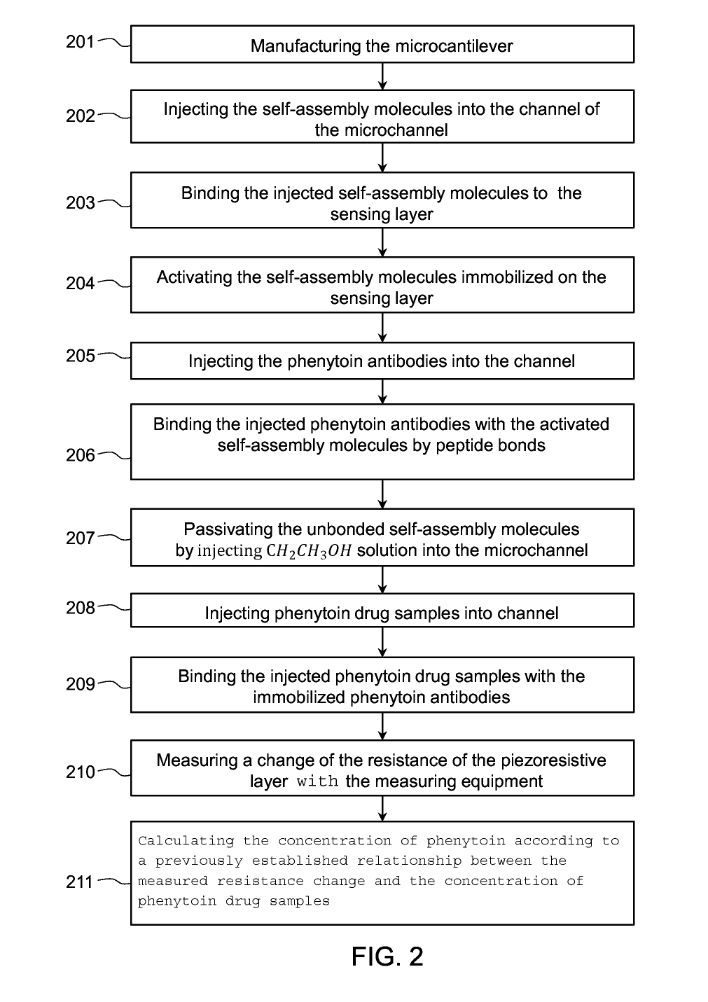

FIG. 2 is a flowchart illustrating one embodiment of a method for measuring the concentration of phenytoin. As shown in FIG. 2, the steps of the method may comprise:

Step 201: Manufacturing the microcantilever 102;

Step 202: Injecting the self-assembly molecules into the channel 132 of the microchannel 108 since the phenytoin antibodies cannot bind directly to the sensing layer 126.

Step 203: The injected self-assembly molecules bind to the sensing layer 126. As a result, the self-assembly monolayer 104 may be formed.

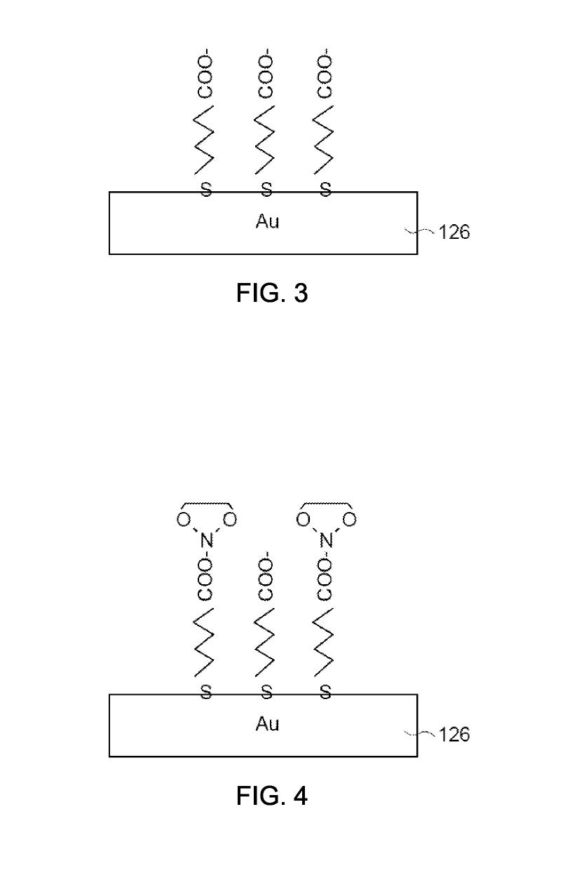

FIG. 3 is a schematic view illustrating the self-assembly molecules and the microcantilever 102. As shown in FIG. 3, the sulfur atoms in self-assembly molecules may bond to and be immobilized on the sensing layer 126 by covalent bonds.

Step 204: FIG. 4 is a schematic view of the activation of the self-assembly molecules. As shown in FIG. 4, the self-assembly molecules immobilized on the sensing layer 126 may be activated. Thus, the self-assembly molecules may be bonded with the phenytoin antibodies.

Step 205: Injecting the phenytoin antibodies into the channel 132.

Step 206: The injected phenytoin antibodies may bind with the activated self-assembly molecules by peptide bonds. Subsequently, the phenytoin antibody layer 106 may be formed.

Step 207: FIG. 5 is a schematic view illustrating the passivating of the self-assembly molecules. Since not all of the self-assembly molecules are bonded with the injected phenytoin antibodies, passivating the unbonded self-assembly molecules by injecting CH₂CH₃OH solution into the microchannel 108 may be necessary. Subsequently, the passivated self-assembly molecules may not be bonded with other molecules.

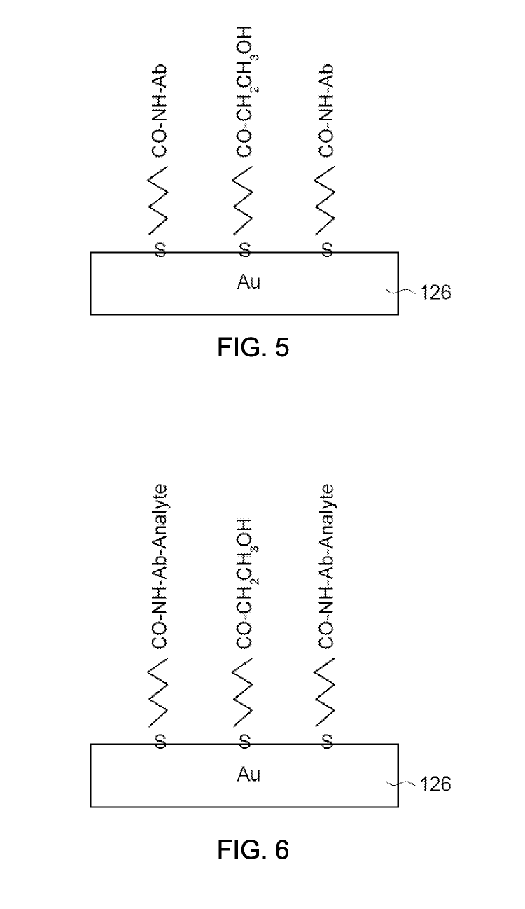

Step 208: FIG. 6 is a schematic view of the immobilized phenytoin antibodies and phenytoin drug samples. Injecting phenytoin drug samples into the channel 132.

Step 209: The injected phenytoin drug samples may bind with the immobilized phenytoin antibodies.

Step 210: Measuring a change of the resistance of the piezoresistive layer 122 with the measuring equipment 110.

Step 211: The concentration of phenytoin may be calculated according to the previously established relationship between the measured resistance change and the concentration of phenytoin drug samples.

One of ordinary skill in the art having the benefit of the instant disclosure would appreciate that the resistance of the microcantilever 102 may be measured and that the surface stress of the piezoresistive layer 122 may be calculated. The measurements and calculations may occur during Steps 202, 204, and 207. Accordingly, one of ordinary skill in the art having the benefit of the instant disclosure may ensure that the immobilized phenytoin antibodies on the phenytoin biosensor 100 and the phenytoin drug samples change the surface stress of the microcantilever 102 and the resistance of the piezoresistive layer 122.

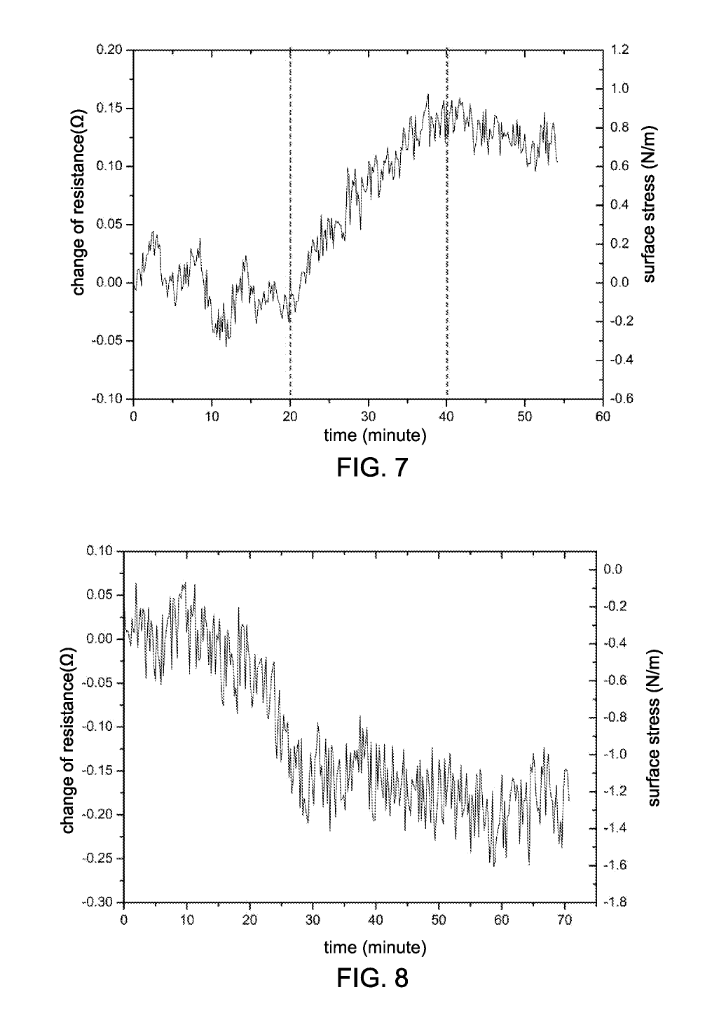

FIG. 7 illustrates the changes in resistance and surface stress after the self-assembly molecules bind to the microcantilever 102. As shown in FIG. 7, when the self-assembly molecules bind to the sensing layer 126 of the microcantilever 102 by covalent bonds, the surface stress of the microcantilever 102 may be changed to 0.8 N/m and the piezoresistive layer 122 may be deformed due to the change of the surface stress. This change in the surface stress may subsequently cause the change of the resistance of the piezoresistive layer 122 by 0.125Ω.

FIG. 8 illustrates the changes in resistance and surface stress after binding the phenytoin antibodies to the microcantilever 102. As shown in FIG. 8, the phenytoin antibodies may bind with the self-assembly monolayer by peptide bonds. Accordingly, the surface stress of the microcantilever 102 may be changed to −1.3 N/m and the piezoresistive layer 122 may be deformed due to the change of the surface stress. This change in the surface stress may cause the change of the resistance of the piezoresistive layer 122 by 0.2Ω.

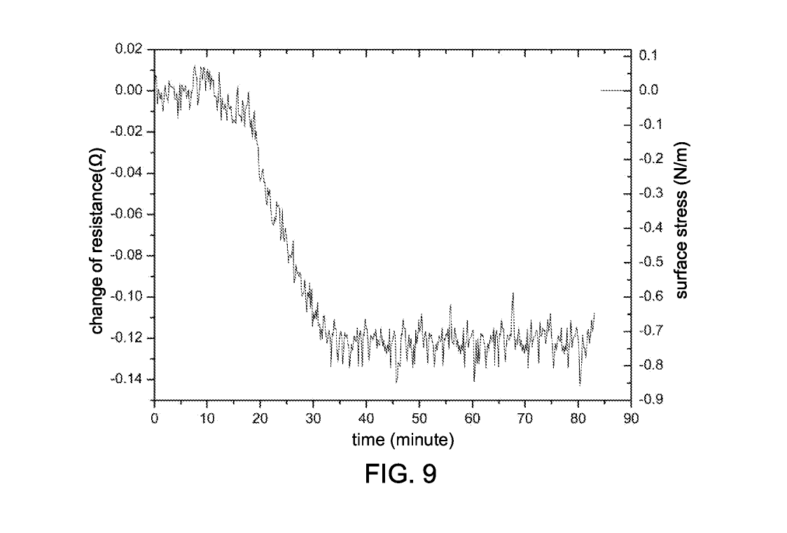

FIG. 9 illustrates the changes in resistance and surface stress after binding the phenytoin drug samples to the microcantilever 102. As shown in FIG. 9, the phenytoin drug samples and the phenytoin antibodies may change the surface stress of the microcantilever 102. The piezoresistive layer 122 may be deformed due to the change in surface stress. The surface stress may be changed to −0.75 N/m, and the resistance of the piezoresistive layer 122 may change by 0.12Ω due to this deformation.

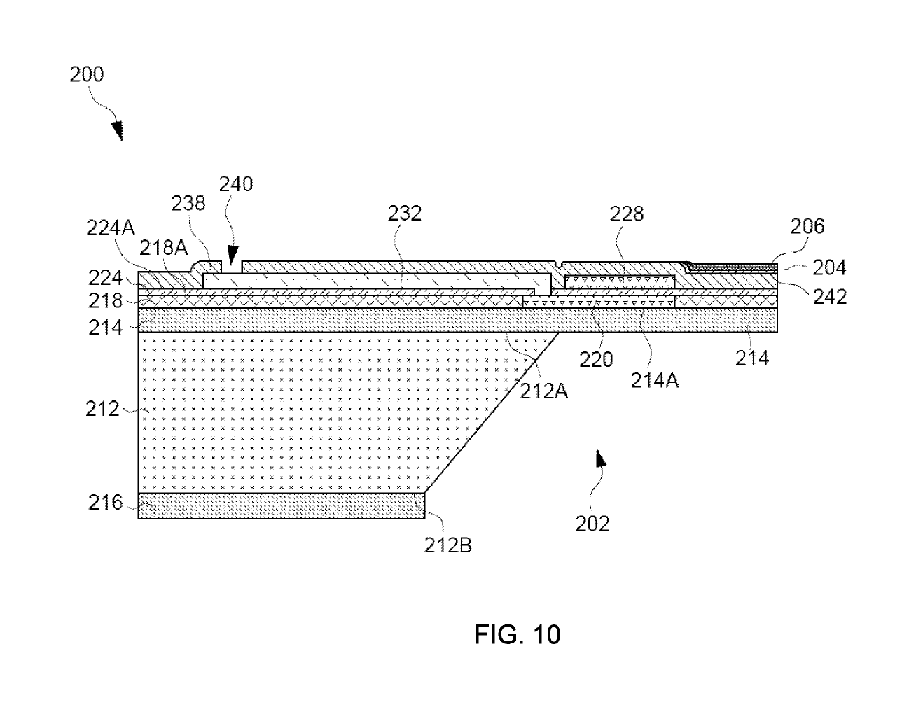

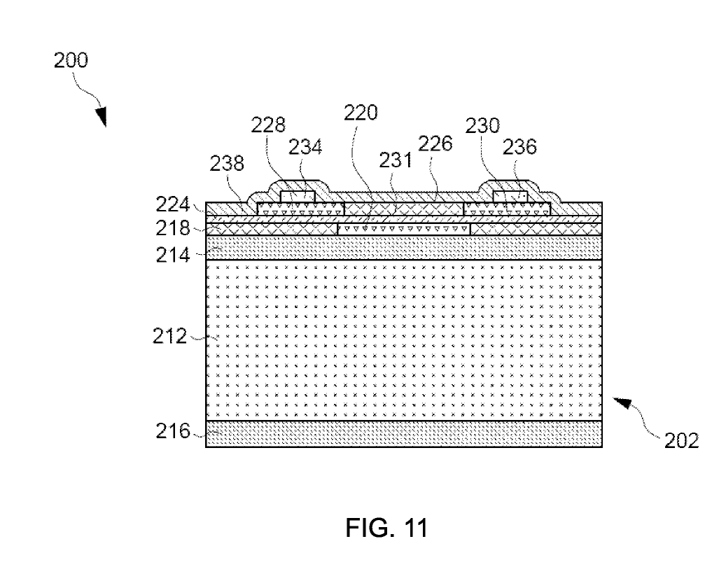

FIGS. 10 and 11 illustrates a side view and front view, respectively, of a phenytoin concentration biosensor 200 according to some example embodiments of the disclosure. In this embodiment, a field effect transistor type microcantilever may replace the piezoresistive type microcantilever in previously described embodiments. Accordingly, the same structures of the two embodiments are not described again as one of ordinary skill in the art would appreciate the other features in light of the previous descriptions in this disclosure. As shown in FIGS. 10 and 11, the phenytoin biosensor 200 may comprise a microcantilever 202, a self-assembly monolayer 204, and a phenytoin antibody layer 206. The microcantilever 202 may include a substrate 212, the substrate 212 may be made of silicon semiconductor doped with Boron or Phosphorous. A back side etching mask 214 may be deposited on an upper surface 212A of the substrate 212, and a lower passivation layer 216 may be deposited on a bottom surface 212B of the substrate 212. The back side etching mask 214 and the lower passivation layer 216 may be made of a Nitride, such as Si₃N₄. A first piezoresistive layer 218 may be deposited on an upper surface 214A of the back side etching mask 214, and the first piezoresistive layer 218 may be made of polysilicon. The first piezoresistive layer 218 may be doped with Phosphorous or Boron to form a gate electrode 220 of a field effect transistor. A dielectric layer 224 of the gate electrode 220 may be deposited on an upper surface 218A of the first piezoresistive layer 218, and the dielectric layer 224 may be made of SiO₂. A second piezoresistive layer 226, which may be made of polysilicon, may be deposited on an upper surface 224A of the dielectric layer 224, and the second piezoresistive layer 226 may be doped with Phosphorous or Boron to form a source electrode 228 and a drain electrode 230 of the field effect transistor. A channel 231 of the field effect transistor may be deposited between the source electrode 228 and the drain electrode 230, and the material of the channel 231 may be different from the materials of the source electrode 228 and the drain electrode 230. The doping material of the second piezoresistive layer 226 may not be directly related to the doping material of the first piezoresistive layer 218. A plurality of conductive wires 232, 234, 236 may be deposited on the upper surface 224A of the dielectric layer 224, and these conductive wires 232, 234, 236 may be in contact with the gate electrode 220, the source electrode 228, and drain electrode 230, respectively. An upper passivation layer 238, which may be made of Nitride, may be deposited on the upper surface 224A of the dielectric layer 224, and the upper passivation layer 238 covers over the second piezoresistive layer 226 and the conductive wires 232, 234, 236. There may be three holes 240 at the end portion of the upper passivation layer 238, and the ends of the conductive wires 232, 234, 236 may be exposed to the holes 240, respectively, to allow for measuring of the electrical signals (the electrical signal may be voltage or current) of field effect transistor. A sensing layer 242 may be deposited on a top surface of the upper passivation layer 238, and the sensing layer 242 may be made of a gold film. In some embodiments, the thickness of the sensing layer 242 may be less than 100 nm.

The self-assembly monolayer 204 may be composed of a plurality of 8-Mercaptooctanoic acid and may bind to the sensing layer 242 of the microcantilever 202. The phenytoin antibody layer 206 may be composed of a plurality of phenytoin antibodies and may bind with the self-assembly monolayer 204. After the injected phenytoin drug samples bind with the phenytoin antibody layer 206, the microcantilever 202 may be deformed. At the same time, the current of the field effect transistor may be changed if the voltages between gate electrode and drain electrode are kept constant. The concentration of the phenytoin may be calculated according the previously established relationship between the change of the current of the field effect transistor and the concentration of the phenytoin drug samples

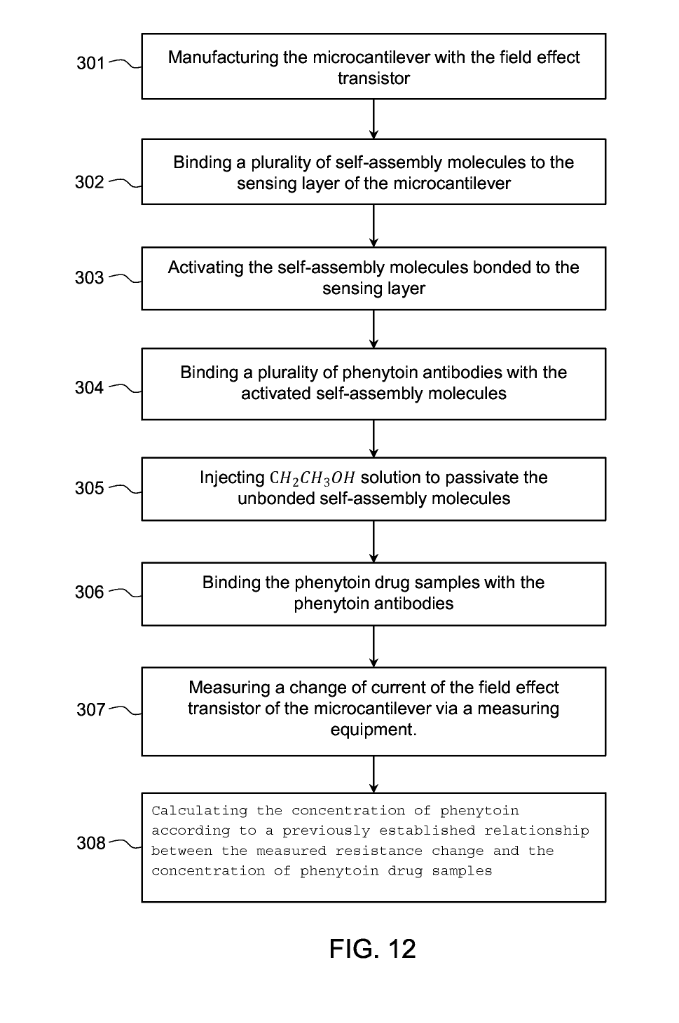

FIG. 12 is a flowchart illustrating another embodiment of a method for measuring the concentration of phenytoin. As shown in FIG. 12, the steps of the method may comprise:

Step 301: Manufacturing the microcantilever 202 with the field effect transistor;

Step 302: A plurality of self-assembly molecules may bind to the sensing layer 242 of the microcantilever 202 since the phenytoin antibodies may not be directly bonded to the sensing layer 242 of the microcantilever 202. Subsequently, the self-assembly monolayer 204 may be formed.

Step 303: Activating the self-assembly molecules bonded to the sensing layer 242, and the self-assembly molecules may easily be bonded with the phenytoin antibodies.

Step 304: Binding a plurality of phenytoin antibodies with the activated self-assembly molecules, so that the phenytoin antibody layer 206 may be formed.

Step 305: Not all of the self-assembly molecules may be bonded with the phenytoin antibodies, injecting CH₂CH₃OH solution to passivate the unbonded self-assembly molecules.

Step 306: Binding the phenytoin drug samples with the phenytoin antibodies.

Step 307: Measuring a change of the current of the field effect transistor of the microcantilever 202 via a measuring equipment.

Step 308: The concentration of phenytoin may be calculated based on the previously determined relationship between the measured current change and the concentration of phenytoin drug samples.

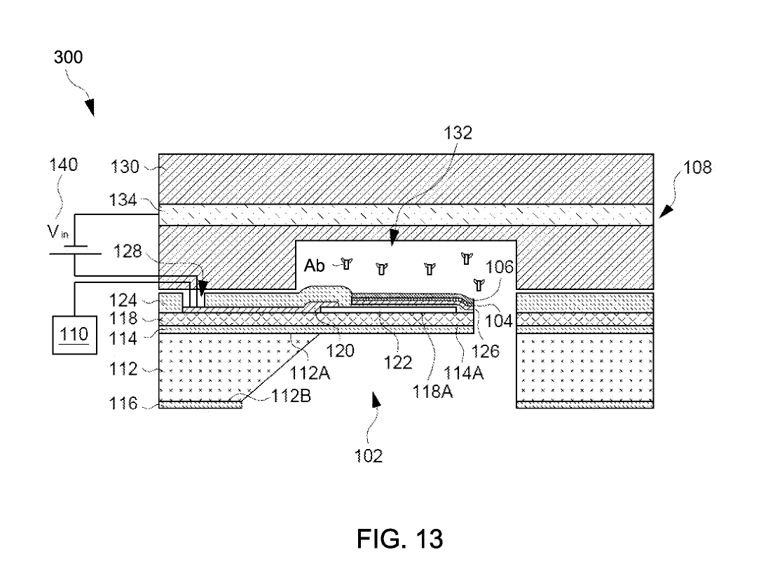

FIG. 13 is a schematic view illustrating a phenytoin biosensor 300 according to some example embodiments of the disclosure. Phenytoin biosensor 300 may further comprise a power supply 140. The cathode and the anode of the power supply 140 may connect with the conductive glass layer 134 and the piezoresistive layer 122, respectively. The power supply 140 may provide negative charges and positive charges to the conductive glass layer 134 and the piezoresistive layer 122, respectively. At the same time, the negative and positive charges may cause an electrical field in the channel 132 and the generated electrical field may point to the surface of microcantilever 102. The generated electrical field may drive more phenytoin antibodies to move toward the microcantilever 102. Thus, more phenytoin antibodies may bind to the microcantilever 102.

One of ordinary skill in the art having the benefit of the instant disclosure would appreciate that the phenytoin biosensor 200 may be connected to the power supply 140. The power supply 140 may provide an electrical field that points to the microcantilever 202, and the generated electrical field may drive more phenytoin drug samples to bind to the microcantilever 202.

One of ordinary skill in the art having the benefit of the instant disclosure would appreciate that the phenytoin biosensor and the method for measuring the concentration of the phenytoin described in the present disclosure may provide for several advantages. For example, the size of the phenytoin biosensor may be sufficiently small to allow for increased portability and may allow for a point-of-care platform and personal diagnosis. As a result, patients may use the biosensor to easily determine whether or not the concentration of the drug in their blood vessels is within the optimal range for effective treatment. As another example, the manufacturing cost for the phenytoin biosensor may be substantially cheaper.

Realizations in accordance with the present disclosure have been described only in the context of particular embodiments. These embodiments are meant to be illustrative and not limiting. Many variations, modifications, additions, and improvements are possible and will become clear to one of ordinary skill in the art. Accordingly, plural instances may be provided for components described herein as a single instance. Structures and functionality presented as discrete components in the exemplary configurations may be implemented as a combined structure or component. These and other variations, modifications, additions, and improvements may fall within the scope of the invention as defined in the claims that follow.

What is claimed is:

1. A phenytoin biosensor comprising:
a microcantilever;
a self-assembly monolayer immobilized on the microcantilever; and
a phenytoin antibody layer immobilized on the self-assembly monolayer, the phenytoin antibody layer operable to bind with phenytoin drug samples.

2. The phenytoin biosensor according to claim 1, further comprising
a microchannel,
wherein the microcantilever is covered in the microchannel,
wherein the microchannel comprises a conductive glass layer, and
wherein the conductive glass layer is disposed above the microcantilever.

3. The phenytoin biosensor according to claim 2, further comprising
a power supply,
a negative electrode of the power supply connected with the glass conductive layer, and
a positive electrode of the power supply connected with a piezoresistive layer of the microcantilever.

4. The phenytoin biosensor according to claim 1, wherein the microcantilever further comprises
a sensing layer, and
a piezoresistive layer,
wherein the self-assembly monolayer is immobilized on the sensing layer, and
wherein the piezoresistive layer is disposed below the sensing layer.

5. The phenytoin biosensor according to claim 4,
wherein the sensing layer comprises a gold film, and
wherein the thickness of the sensing layer is less than 100 nm.

6. The phenytoin biosensor according to claim 4,
wherein the piezoresistive layer comprises polysilicon.

7. The phenytoin biosensor according to claim 1,
wherein the self-assembly monolayer comprises a plurality of self-assembly molecules, wherein the self-assembly molecules are 8-Mercaptooctanoic acid.

8. The phenytoin biosensor according to claim 1,
wherein the microcantilever comprises a field effect transistor and a sensing layer,
wherein the self-assembly monolayer is immobilized on the sensing layer, and
wherein the sensing layer is disposed above the field effect transistor.

9. A method for measuring a concentration of phenytoin, comprising:
manufacturing a microcantilever with a piezoresistive layer;
binding a plurality of self-assembly molecules to the microcantilever;
activating the bonded self-assembly molecules;
binding a plurality of phenytoin antibodies with the activated self-assembly molecules;
binding a plurality of phenytoin drug samples with the phenytoin antibodies;
measuring a change of resistance of the piezoresistive layer; and
calculating the concentration of the phenytoin according to a pre-determined relationship between the measured resistance change and the concentration of the phenytoin drug samples.

10. The method according to claim 9, wherein the method further comprises providing an electrical field, and wherein the electrical field points to the microcantilever.

11. The method according to claim 9, wherein the method further comprises passivating the unbonded self-assembly molecules.

12. The method according to claim 9, wherein the method further comprises passivating the unbonded self-assembly molecules via injecting a CH₂CH₃OH solution.

13. A method for measuring a concentration of phenytoin, comprising:
manufacturing a microcantilever with a field effect transistor;
binding a plurality of self-assembly molecules to the microcantilever;
activating the bonded self-assembly molecules;
binding a plurality of phenytoin antibodies with the activated self-assembly molecules;
binding a plurality of phenytoin drug samples with the phenytoin antibodies;
measuring a change of current of the field effect transistor; and
calculating the concentration of phenytoin according to a pre-determined relationship between the measured current change and the concentration of the phenytoin drug samples.

14. The method according to claim 13 further comprising providing an electrical field, wherein the electrical field points to the microcantilever.

15. The method according to claim 13 further comprising passivating the unbonded self-assembly molecules to prevent the self-assembly molecules which are not bonded with the phenytoin antibodies from binding with other molecules.

16. The method according to claim 15, wherein the passivating the unbonded self-assembly molecules further comprises injecting a CH₂CH₃OH solution.
